SEQUENCE LISTING (14 sequences)

SEQ ID NO: 1 - DNA, 23 bp, Artificial
  Primer
tcctgctgtgcttctccaccaca

SEQ ID NO: 2 - DNA, 23 bp, Artificial
  Primer
aagtccgccctgtaggtgaggtt

SEQ ID NO: 3 - DNA, 25 bp, Artificial
  Primer
acagcaaccatgggagagaatgctg

SEQ ID NO: 4 - DNA, 23 bp, Artificial
  Primer
acgtaggccaggaggttgtgcat

SEQ ID NO: 5 - DNA, 22 bp, Artificial
  Primer
atcatccctgcgagcctatcct

SEQ ID NO: 6 - DNA, 24 bp, Artificial
  Primer
gaccttttttggctaaacgctttc

SEQ ID NO: 7 - DNA, 23 bp, Artificial
  Primer
tcctgctgtgcttctccaccaca

SEQ ID NO: 8 - DNA, 23 bp, Artificial
  Primer
aagtccgccctgtaggtgaggtt

SEQ ID NO: 9 - DNA, 25 bp, Artificial
  Primer
acagcaaccatgggagagaatgctg

SEQ ID NO: 10 - DNA, 23 bp, Artificial
  Primer
acgtaggccaggaggttgtgcat

SEQ ID NO: 11 - DNA, 22 bp, Artificial
  Primer
atcatccctgcgagcctatcct

SEQ ID NO: 12 - DNA, 24 bp, Artificial
  Primer
gaccttttttggctaaacgctttc

SEQ ID NO: 13 - DNA, 21 bp, Artificial
  Primer
acggccgcatcttcttgtgca

SEQ ID NO: 14 - DNA, 21 bp, Artificial
  Primer
acggccaaatccgttcacacc

(19) United States
(12) Patent Application Publication
(10) Pub. No.: US 2021/0346401 A1
(43) Pub. Date: Nov. 11, 2021

(54) REGULATOR OF TGR5 SIGNALING AS IMMUNOMODULATORY AGENT

(71) Applicant: YICHANG HUMANWELL PHARMACEUTICAL CO., LTD, Yichang, Hubei (CN)

(72) Inventors: Hongbing SHU, Wuhan (CN); Mingming HU, Wuhan (CN)

(73) Assignee: YICHANG HUMANWELL PHARMACEUTICAL CO., LTD, Yichang, Hubei (CN)

(21) Appl. No.: 17/277,474

(22) PCT Filed: Sep. 25, 2019
(86) PCT No.: PCT/CN2019/107719

§ 371 Date: Mar. 18, 2021

(30) Foreign Application Priority Data
Sep. 25, 2018 (CN) PCT/CN2018/107358

(51) Int. Cl. A61K 31/575; A61K 39/39

(52) U.S. Cl. CPC A61K 31/575; A61K 2039/55511; A61K 39/39

(57) ABSTRACT

The present disclosure provides methods of enhancing immunity, comprising administering TGR5 agonist such as a bile acid (BA) to a subject in need thereof. Provided also herein are methods of inhibiting immunity, comprising administering TGR5 antagonist to a subject in need thereof. The present disclosure further provides methods of identifying immunomodulatory agent, comprising contacting TGR5 with a candidate agent.

This application claims the priority of PCT Application No. PCT/CN2018/107358, filed on Sep. 25, 2018, and titled with “Regulator of TGR5 Signaling as Immunomodulatory Agent”, and the disclosures of which are hereby incorporated by reference.

### FIELD OF INVENTION

The present disclosure provides methods of enhancing immunity, comprising administering TGR5 agonist such as a bile acid (BA) to a subject in need thereof. Provided also herein are methods of inhibiting immunity, comprising administering TGR5 antagonist to a subject in need thereof. The present disclosure further provides methods of identifying immunomodulatory agent, comprising contacting TGR5 with a candidate agent.

### BACKGROUND OF THE INVENTION

Bile acids (BAs) are steroid acids found predominantly in the bile of mammals and other vertebrates. Primary bile acids are those synthesized by the liver, while secondary bile acids result from bacterial actions in the colon. Conjugated bile acids are those conjugated with taurine or glycine, while free bile acids correspond to bile acids which are not conjugated.

The concentration of bile acids/salts in the small intestine is high enough to form micelles and solubilize lipids. Bile acid-containing micelles aid lipases to digest lipids and bring them near the intestinal brush border membrane, which results in fat absorption.

Bile acids have other functions, including eliminating cholesterol from the body, driving the flow of bile to eliminate certain catabolites (including bilirubin), emulsifying fat-soluble vitamins to enable their absorption, and aiding in motility and the reduction of the bacteria flora found in the small intestine and biliary tract.

Y. Calmus and coworkers found, chenodeoxycholic acid and ursodeoxycholic acid inhibits the production of interleukin-1, interleukin-6 and tumor necrosis factor-α, and exerts more or less strong immune-inhibitory effects on monocyte activity in vitro, which is believed to be mediated by TGR5 activation (Calmus Y, Guechot J, Podevin P, Bonnefis M T, Giboudeau J, Poupon R. Differential effects of chenodeoxycholic and ursodeoxycholic acids on interleukin1, interleukin 6 and tumor necrosis factor-alpha production by monocytes. Hepatology 1992; 16:719-23.[15]).

The anti-inflammatory effect of TGR5 is mediated by the inhibition of the pro-inflammatory transcriptional nuclear factor-κB (NF-κB) (Pols T W H, Nomura M, Harach T, et al. TGR5 activation inhibits atherosclerosis by reducing macrophage inflammation and lipid loading. Cell Metabolism 2011; 14:747-57; Wang Y-D, Chen W-D, Yu D, Forman B M, Huang W. The G-protein-coupled bile acid receptor, Gpbar1 (TGR5), negatively regulates hepatic inflammatory response through antagonizing NF-κB in mice. Hepatology 2011; 54:1421-32). In macrophages from TGR5 knockout mice, the mRNA levels of various pro-inflammatory genes targeted by NF-κB (inducible NOS, interferon-inducible protein, and IL-la) are higher than to those in macrophages from wild-type mice. Likewise, in the macrophage cell line RAW264.7, TGR5 activation inhibited NF-κB activation. When TGR5 was activated or overexpressed, NF-κB transcriptional activity was inhibited after LPS treatment.

However, the present inventors surprisingly found that bile acids enhanced innate immune response, then found a novel immunomodulatory role of TGR5-GRK-β-arrestin-SRC signaling which is different from the prior art knowledge, and accordingly completed the present invention.

### SUMMARY OF THE INVENTION

The present invention is based on unexpected founding of the present inventors that bile acids enhanced innate immune response.

Infection of viruses causes intracellular accumulation of bile acids, e.g. chenodeoxycholic acid (CDCA), lithocholic acid (LAC) or deoxycholic acid (DCA).

Bile acids, e.g. CDCA, LAC and DCA, act as agonists of TGR5, which activates downstream signaling components GRKs, β-arrestins and SRC.

Bile acids and generally agonists of TGR5-GRK-β-arrestin-SRC induce immune response, which is able to clear viruses.

In the first aspect, the present disclosure provides a method of enhancing immunity in a subject in need thereof, comprising:

administering a TGR5-GRK-β-arrestin-Src agonist to the subject.

In an embodiment, the immunity is an immunity against microbial infection, e.g., bacterial infection, viral infection, fungal infection.

In a particular embodiment, the viral infection is caused by DNA virus or RNA virus such as ssDNA virus, dsDNA virus, ssRNA virus or dsRNA virus, e.g. virus selected from the group consisting of herpes simplex virus (HSV) including HSV-1 and HSV-2, human cytomegalovirus (HCMV), Kaposi's sarcoma-associated herpes virus (KSHV), hepatitis B virus (HBV), hepatitis C virus (HCV), Zika Virus, EV71, influenza virus, Human Immunodeficiency Virus (HIV), EB virus, and human papillomavirus (HPV).

In another embodiment, the microbial infection induces a disease, which is for example selected from the group consisting of tuberculosis, candidiasis, aspergillosis, alginosis, nocardia and cryptococcosis.

In another embodiment, the immunity is an immunity against tumor, e.g., solid tumor or leukemia.

In a further embodiment, the TGR5-β-arrestin-Src agonist is TGR5 agonist, GRK agonist, β-arrestin agonist, and/or Src agonist,

wherein the GRK agonist is for example an agonist of GRK1, GRK2, GRK3, GRK4, GRK5, and/or GRK6, particularly of GRK2, GKR4, and/or GRK6, more particularly of GRK6, and

wherein the β-arrestin agonist is for example an agonist of β-arrestin-1 and/or β-arrestin-2.

In another embodiment, the TGR5-GRK-β-arrestin-Src agonist is a bile acid source, particularly a bile acid, e.g. primary bile acid or secondary bile acid, unconjugated bile acid or conjugated bile acid.

In another embodiment, the bile acid is selected from cholic acid (CA), chenodeoxycholic acid (CDA), deoxycholic acid (DCA), lithocholic acid (LCA), glycocholic acid, taurocholic acid, glycochenodeoxycholic acid, taurochenodeoxycholic acid, ketolithocholic acid, sulpholithocholic acid, ursodeoxycholic acid, glycodeoxycholic acid, taurodeoxycholic acid, glycolithocholic acid, taurolithocholic acid, and any combination thereof.

In a second aspect, the present disclosure provides a method of inhibiting immunity in a subject in need thereof, comprising:

administering a TGR5-GRK-β-arrestin-Src antagonist to the subject.

In an embodiment, the immunity is associated with autoimmune disease.

In another embodiment, the TGR5-GRK-β-arrestin-Src antagonist is TGR5 antagonist, β-arrestin-1/2 antagonist, and/or Src antagonist.

In a third aspect, the present disclosure provides a method of treating disease treatable by modulating immunity in a subject in need thereof, comprising administering a TGR5-GRK-β-arrestin-Src modulatory agent to the subject.

In an embodiment, the disease is selected from the group consisting of microbial infection including viral infection, bacterial infection and fungal infection, tumor including solid tumor and leukemia, and the TGR5-GRK-β-arrestin-Src modulatory agent is TGR5-GRK-β-arrestin-Src agonist, e.g., TGR5 agonist, β-arrestin-1/2 agonist, and/or Src agonist.

In another embodiment, the disease is autoimmune disease, and the TGR5-GRK-β-arrestin-Src modulatory agent is TGR5-GRK-β-arrestin-Src antagonist, e.g., TGR5 antagonist, β-arrestin-1/2 antagonist, and/or Src antagonist.

In a fourth aspect, the present disclosure provides a method of vaccinating a subject in need thereof, comprising administering to the subject TGR5-GRK-β-arrestin-Src agonist, e.g., TGR5 agonist, β-arrestin-1/2 agonist, and/or Src agonist, as adjuvant separately or in a vaccine composition.

Correspondingly the present disclosure provides a vaccination adjuvant composition, comprising TGR5-GRK-β-arrestin-Src agonist, e.g., TGR5 agonist, β-arrestin-1/2 agonist, and/or Src agonist. And the present disclosure further provides a vaccine composition, comprising TGR5-GRK-β-arrestin-Src agonist, e.g., TGR5 agonist, β-arrestin-1/2 agonist, and/or Src agonist, as adjuvant.

In a fifth aspect, the present disclosure provides a method of identifying immune modulatory agent, comprising

contacting a candidate agent with reporter of TGR5-GRK-β-arrestin-Src pathway, and

determining activity of TGR5-GRK-β-arrestin-Src pathway,

wherein changed activity of TGR5-GRK-β-arrestin-Src pathway indicates the candidate agent is an immune modulatory agent.

In an embodiment, enhanced activity of TGR5-GRK-β-arrestin-Src pathway indicates the candidate agent is an immune enhancer, and reduced activity of TGR5-GRK-β-arrestin-Src pathway indicates the candidate agent is an immune inhibitor.

### DESCRIPTION OF THE DRAWINGS

FIG. 1.

Viral infection induces expression of several proteins involved in biosynthesis and absorption of BAs via NF-κB-dependent manner.

A. Heatmap of induction of gene expression involved in BA metabolism by viral infection. THP1 cells were infected with HSV-1 or SeV for the indicated times, followed by qPCR analysis of expression of the indicated genes, and then data were subjected to analysis by Heatmap.

B. Effects of different inhibitors on virus-induced expression of several genes involved in BA metabolism. THP1 cells were treatment with DMSO or the indicated inhibitors, followed by infection with HSV-1 or SeV for 4 hours before qPCR analysis of expression of the indicated genes.

C. Effects of IRF3- or p65-knockout on SeV-induced expression of proteins involved in BA metabolism. THP1 cells stably transduced with control, gRNA of IRF3 or p65 were infected with SeV for the indicated times before qPCR analysis of the indicated genes.

D. Effects of IRF3- or p65-knockout on HSV-1-induced expression of proteins involved in BA metabolism. Experiments were similarly performed as C, except that HSV-1 was used instead of SeV.

E. CHIP-analysis of p65 binding to the promoters of the indicated genes. THP1 cells were infected with SeV or HSV-1 for the indicated times before CHIP assay were performed, followed by qPCR analysis of the abundance of p65-bounded DNA fragments with the indicated primers.

F. Effects of VISA-knockout on viral infection-induced expression of proteins involved in BA metabolism. THP1 cells stably transduced with control and gRNA of VISA were infected with SeV for the indicated times before qPCR analysis of the indicated genes.

G. Effects of MITA-knockout on viral infection-induced expression of proteins involved in BA metabolism. THP1 cells stably transduced with control and gRNA of MITA were infected with HSV-1 for the indicated times before qPCR analysis of the indicated genes.

H. Effects of VISA-knockout on viral infection-induced phosphorylation of IKKα/β, p65 and IRF3. THP1 cells stably transduced with control and gRNA of VISA were infected with SeV for the indicated times, and cells were lysed for immunoblotting analysis with the indicated antibodies.

I. Effects of MITA-knockout on viral infection-induced phosphorylation of IKKα/β, p65 and IRF3. THP1 cells stably transduced with control and gRNA of MITA were infected with HSV-1 for the indicated times, and cells were lysed for immunoblotting analysis with the indicated antibodies.

J. Viral infection-induced expression of proteins involved in BA metabolism. THP1 cells were infected with HSV-1 or SeV for the indicated times, followed by qPCR analysis of the expression of the indicated genes.

K. Effect of UV treatment on virus-induced expression of genes involved in BA metabolism. THP-1 cells were infected with the wild-type or UV-treated HSV-1 and SeV for the indicated times before qPCR analysis of the expression of the indicated genes.

L. Effect of UV treatment on virus-induced phosphorylation of IRF3, IKKα/β and p65. THP-1 cells were infected with the wild-type or UV-treated HSV-1 and SeV for the indicated times before immunoblotting analysis with the indicated antibodies.

FIG. 2.

Viral infection induces accumulation of intracellular bile acids via both biosynthesis and absorption.

A. Measurement of intracellular BA in THP-1 cells cultured in complete and blank medium. THP-1 cells were cultured in refresh complete medium (CM) or blank medium (BM) and then infected with SeV or HSV-1 for the indicated times before cells were harvested for measurement of the BA.

B. Measurement of intracellular BA in hepatic cells cultured in complete and blank medium. Experiments were similarly performed as (A), except that hepatic WRL68 cells were used.

C. Analysis of intracellular BAs in in primary mouse hepatocytes cultured in complete medium (CM) or blank medium by MS. Primary mouse hepatocytes were cultured in complete or blank medium (BM) for 24 hours, and then infected with SeV or HSV-1 for the indicated times before cells were harvested for analysis of intracellular BAs by MS.

D. Effects of knockdown of OATP, CYP7A1, CYP7B1 or HSD3B7 on virus-induced accumulation of intracellular BAs. THP1 cells stably transduced with control, shRNA of OATP, CYP7A1, CYP7B1 or HSD3B7 were infected with HSV-1 or SeV for 6 hours before cells were harvested for measurement of the TBA.

E. Measurement of intracellular BA in HEK293 cells cultured in complete and blank medium. HEK293 cells were cultured in refresh complete medium (CM) or blank medium (BM) and then infected with SeV for the indicated times before cells were harvested for measurement of BA.

FIG. 3.

Virus-triggered accumulation of intracellular BAs promotes type I IFNs production and antiviral innate immune responses.

A. Effects of knockdown of OATP, CYP7A1, CYP7B1, CYP27A1 and HSD3B7 individually on virus-triggered expression of IFNB1 and CXCL10. THP1 cells stably transduced with control or shRNA of OATP, CYP7A1, CYP7B1, CYP27A1 or HSD3B7 were infected with HSV-1 or SeV for 8 hours before qPCR analysis of the expression of the indicated genes.

B. Effects of knockdown of OATP, CYP7A1, CYP7B1, CYP27A1 and HSD3B7 individually on IFN-γ-induced expression of IRF1. Experiments were performed as A, except that IFN-γ were treated for 8 hours before qPCR analysis of the expression of the indicated genes.

C. Effects of knockdown of OATP, CYP7A1 and CYP7B1 individually on virus-triggered phosphorylation of IRF3. THP1 cells stably transduced with control or shRNA of OATP, CYP7A1 or CYP7B1 were infected with HSV-1 or SeV for 4 hours before immunoblotting analysis with the indicated antibodies.

D. Induction of type I IFNs and other antiviral genes by BAs. Raw264.7 cells were treated by the indicated concentrations of the indicated BAs, followed by qPCR analysis of the expression of the indicated genes.

E. Induction of phosphorylation of TBK1 and IRF3 by BAs. Raw264.7 cells were treated by the indicated concentrations of CDCA and LCA, followed by immunoblotting analysis with the indicated antibodies.

F. Effects of BAs on virus-induced expression of type I IFNs and other antiviral genes. Raw264.7 cells were infected with HSV-1 or SeV for half an hour before treatment with LCA or CDCA (100 μM), followed by immunoblotting analysis with the indicated antibodies.

G. Effects of BAs on virus-induced phosphorylation of IRF3. Raw264.7 cells were infected with HSV-1 or SeV for half an hour before treatment with CDCA (100 μM), followed by immunoblotting analysis with the indicated antibodies.

H. Effects of BA on viral replication. RAW264.7 cells were infected with VSV-GFP for 1 hours before replacement of the medium containing DMSO or the indicated concentrates of CDCA, and then cells were cultured for another 24 hours, followed by fluorescence microscope experiments and flow cytometry analysis were performed.

I. Schematic diagram for BA biosynthesis pathway in liver.

J. Effects of knockdown of many enzymes involved in BA biosynthesis pathway individually on virus- as well as transfected RNA- and DNA-induced activation of IFN-β in reporter assays. HEK293 cells were transfected with the indicated plasmids for 36 hours before viral infection for 10 hours or transfected RNA or DNA for 18 hours, and then cells were lysed for luciferase assays.

K. Effects of knockdown of many enzymes involved in BA biosynthesis pathway individually on IFN-γ-induced activation of IRF1 in reporter assays. HEK293 cells were transfected with the indicated plasmids for 36 hours before IFN-γ stimulation for 10 hours, and then cells were lysed for luciferase assays.

L. Induction of type I IFNs and other antiviral genes by BAs. Raw264.7 cells were treated with LCA (200 μM) or CDCA (200 μM) for the indicated times, followed by qPCR analysis of the expression of the indicated genes.

M. Effects of BA on viral replication. RAW264.7 cells were infected with VSV-GFP for 1 hours before replacement of the medium containing DMSO or the indicated concentrates of CDCA, and then cells were cultured for another 24 hours, followed by flow cytometry analysis were performed.

FIG. 4.

TGR5-GRK-β-arrestin-SRC pathway is activated in response to viral infection.

A. Effects of knockdown of TGR5 or FXR on virus-induced activation of IFN-β and IFN-γ-induced activation of IRF1 in reporter assays. HEK293 cells were transfected with the indicated plasmids together with the reporter plasmid for 36 hours before infection with SeV or treatment with IFN-γ for 12 hours, and then cells were lysed for luciferase assays. The knockdown efficiency of the indicated plasmids were showed on the left panel.

B. Effects of Tgr5-deficiency on virus-induced expression of Ifnb1 and Cxcl10. Tgr5^{+/+} and Tgr5^{-/-} BMDMs were infected with the indicated viruses for 6 hours before qPCR analysis of the expression of the indicated genes.

C. Effects of Tgr5-deficiency on virus-induced phosphorylation of TBK1 and IRF3. Tgr5^{+/+} and Tgr5^{-/-} BMDMs were infected with HSV-1 or SeV for the indicated times before immunoblotting analysis with the indicated antibodies.

D. Effects of CDCA and INT-777 on viruses-induced expression of Ifnb1 in Tgr5^{+/+} and Tgr5^{-/-} cells. Tgr5^{+/+} and Tgr5^{-/-} BMDMs were infected with HSV-1 or SeV for half an hour, followed by CDCA or INT-777 treatment for another 6 hours before cells were harvested for qPCR analysis of expression of Ifnb1.

E. Effects of ARRB1/2-deficiency on virus-induced expression of IFNB1 and CXCL10. THP1 cells stably transduced with the control or gRNA of ARRB1/2 were infected with SeV or HSV-1 for 8 hours before qPCR analysis of the expression of the indicated genes.

F. Effects of knockdown of GRKs on virus-triggered activation of IFN-β. HEK293 cells were transfected with the indicated plasmids before infection with SeV for 12 hours, and then cells were lysed for luciferase assays.

G. Induction of pY416 of SRC by INT-777 and BA treatments. HEK293 cells were treated with DMSO, INT-777 or the indicated BAs for 1 hours, followed by immunoblotting analysis with the indicated antibodies.

H. Effect of TGR5-deficiency on viruses-induced p-SRC at Y416. Tgr5^{+/+} and Tgr5^{-/-} BMDMs were infected with HSV-1 or SeV for the indicated times before immunoblotting analysis with the indicated antibodies.

T. Association of β-arrestin1/2, GRK6 and SRC with TGR5 induced by viral infection. HEK293 cells were infected with SeV for the indicated times before cells were lysed for coimmunoprecipitation with pre-immunized serum or TGR5 antibodies, and then the immunoprecipitates and lysates were subjected to immunoblotting analysis with the indicated antibodies.

J. Effects of SRC-deficiency on virus-induced expression of IFNB1 and CXCL10. MLFs stably transduced with the control or gRNA of SRC were infected with SeV or HSV-1 for 8 hours before qPCR analysis of the expression of the indicated genes.

K. Effects of SRC-deficiency on virus-induced phosphorylation of TBK1 and IRF3. MLFs stably transduced with the control or gRNA of SRC were infected with SeV for the indicated times before immunoblotting analysis with the indicated antibodies.

L. Effects of Tgr5-deficiency on transfected nucleotides- and cGAMP-induced expression of Ifnb1 and Cxcl10. Tgr5^{+/+} and Tgr5^{-/-} BMDMs were transfected with the indicated nucleotides or cGAMP for 4 hours, and then cells were harvested for qPCR analysis of the expression of the indicated genes.

M. Induction of type I IFNs and other antiviral genes by INT-777. Raw264.7 cells were treated with INT-777 for the indicated concentrations, followed by qPCR analysis of the expression of the indicated genes.

N. Effects of G proteins- or β-arrestin-deficiency on virus-triggered activation of IFN-β. HEK293 cells were transfected with the indicated plasmids together with the reporter plasmid for 36 hours before SeV infection for 10 hours, and then cells were lysed for luciferase assays. The knockdown efficiency of the indicated plasmids were showed in the left panel.

O. Examination of the knockdown efficiency of GRKs-shRNA. HEK293 cells were transfected with the indicated plasmids for 48 hours, followed by qPCR analysis of the expression of the indicated genes.

P. Effects of G proteins, β-arrestin- or GRKs-deficiency on IFN-γ-induced activation of IRF1. HEK293 cells were transfected with the indicated plasmids together with the reporter plasmid for 36 hours before IFN-γ treatment for 10 hours, and then cells were lysed for luciferase assays.

Q. Effects of β-arrestin- or GRKs-deficiency on virus-induced phosphorylation of TBK1 and IRF3. HEK293 cells were transfected with the indicated plasmids for 36 hours before SeV infection for the indicated times, followed by immunoblotting analysis with the indicated antibodies.

R. Effects of β-arrestin1/2-deficiency on virus-induced pY416 of SRC. HEK293 cells were transfected with the indicated plasmids for 36 hours before SeV infection for the indicated times, followed by immunoblotting analysis with the indicated antibodies.

S. Effects of SRC-deficiency on virus-triggered activation of IFN-β. HEK293 cells were transfected with the indicated plasmids together with the reporter plasmid for 36 hours before SeV infection for 10 hours, and then cells were lysed for luciferase assays. The knockdown efficiency of the indicated plasmids were showed in the left panel.

T. Effects of SRC-deficiency on IFN-γ-induced activation of IRF1. HEK293 cells were transfected with the indicated plasmids together with the reporter plasmid for 36 hours before IFN-γ treatment for 10 hours, and then cells were lysed for luciferase assays.

FIG. 5.

SRC mediates global tyrosine phosphorylation of multiple components of antiviral signaling

A. Interaction of SRC with multiple proteins in antiviral signaling in overexpressed system. HEK293 cells were transfected with the indicated plasmids for 24 hours before cells were lysed for coimmunoprecipitation with IgG or HA antibody, and then the immunoprecipitates and lysates were subjected to immunoblotting analysis with the indicated antibodies.

B. Endogenous association of SRC with multiple proteins in antiviral signaling induced by viral infections. MLFs were infected with SeV or HSV-1 for the indicated times before cells were lysed for coimmunoprecipitation with IgG or SRC antibody, and then the immunoprecipitates and lysates were subjected to immunoblotting analysis with the indicated antibodies.

C. Effects of SRC on tyrosine phosphorylation of multiple proteins in antiviral signaling. HEK293 cells were transfected with the indicated plasmids for 24 hours before cells were lysed for immunoprecipitation with HA antibody, and then the immunoprecipitates and lysates were subjected to immunoblotting analysis with the indicated antibodies.

D. Effects of SRC-deficiency on virus-induced tyrosine phosphorylation of multiple proteins in antiviral signaling. MLFs stably transduced with the control or gRNA of SRC were infected with SeV or HSV-1 for the indicated times before cells were lysed for immunoprecipitation with the indicated antibodies, and then the immunoprecipitates and lysates were subjected to immunoblotting analysis with the indicated antibodies.

E-H. Identification of SRC-targeted tyrosine phosphorylation sites of multiple proteins in antiviral signaling. HEK293 cells were transfected with the indicated plasmids for 24 hours before cells were lysed for immunoprecipitation with the indicated antibodies, and the immunoprecipitation were subjected to immunoblotting with the indicated antibodies.

I. Activation of ISRE by the wild-type proteins and their mutants in antiviral signaling. HEK293 cells were transfected with the indicated plasmids together with the reporter plasmid for 24 hours, and then cells were lysed for luciferase assays.

J. Prediction of SRC-targeted phosphorylation sites of multiple proteins in antiviral signaling by the program of GPS 3.0.

FIG. 6.

BAs-TGR5 pathway is critical for host defense against viral infection in vivo.

A. Effects of Tgr5-deficiency on virus-induced expression of serum cytokines. Tgr5^{+/+} and Tgr5^{-/-} mice (n=8) were infected i.p. with HSV-1 or EMCV at 1×10⁷ pfu per mouse for 6 hours before serum cytokines were measured by ELISA.

B. Measurements of viral titers in the brain of Tgr5^{+/+} and Tgr5^{-/-} mice. Tgr5^{+/+} and Tgr5^{-/-} mice (n=6) were infected i.p. with HSV-1 at 1×10⁷ pfu per mouse or EMCV at 1×10⁶ pfu per mouse, and brains were retrieved 3 days later for viral load measurement.

C. Survival rates of Tgr5^{+/+} and Tgr5^{-/-} mice following viral infections. Tgr5^{+/+} and Tgr5^{-/-} mice (n=12) were infected i.p. with HSV-1 at 1×10⁷ pfu per mouse or EMCV at 1×10⁶ pfu per mouse, and the survival rates of mice were observed and recorded for two weeks.

D. Effects of CDCA on viral infection-induced production of serum cytokines of Tgr5^{+/+} and Tgr5^{-/-} mice. Tgr5^{+/+} and Tgr5^{-/-} mice were infected with HSV-1 at 1×10⁷ pfu per mouse for an hour, followed by intraperitoneal injection with CDCA (30 g/kg), and then 6 hours later, serum cytokines were measured by ELISA.

E. Effects of CDCA on viral infection-induced death of mice. Tgr5^{+/+} and Tgr5^{-/-} mice were infected with HSV-1 at 5×10⁷ pfu per mouse for an hour, followed by intraperitoneal injection with CDCA (30 g/kg), and then survival rates were recorded for two weeks.

F. A work model for the regulation of antiviral innate immunity by BA metabolism.

### DETAILED DESCRIPTION OF THE INVENTION

### Definitions

The term “bile acids (BAs)” are steroid acids found predominantly in the bile of mammals and other vertebrates. Different molecular forms of bile acids can be synthesized in the liver by different species.

Primary bile acids, including cholic acid (CA) and chenodeoxycholic acid (CDCA), are those synthesized by the liver of an animal. Secondary bile acids, including deoxycholic acid (DCA) and lithocholic acid (LCA), result from bacterial actions in the intestine. It shall be understood that primary and secondary bile acids are merely classified by their locations of synthesis. A particular primary bile acid can be produced in another location than the liver of an animal, while a particular secondary bile acid can be produced from another than bacterial action, and in another location than the intestine.

Bile acids can be alternatively classified into free bile acids and conjugated bile acids. Free bile acids are those which are in the original form after in situ synthesis, include CA, CDCA, DCA and LCA. Conjugated bile acids are conjugates of free bile acids with taurine or glycine, include taurocholic acid and glycocholic acid (derivatives of cholic acid), and taurochenodeoxycholic acid and glycochenodeoxycholic acid (derivatives of chenodeoxycholic acid), and also taurine and glycine conjugates of DCA and LCA. Bile acids are generally in salt form thereof, mainly potassium salts and sodium salts. These bile acids in salt form are generally called bile salts.

In humans, taurocholic acid and glycocholic acid (derivatives of cholic acid) and taurochenodeoxycholic acid and glycochenodeoxycholic acid (derivatives of chenodeoxycholic acid) are the major bile salts in bile and are roughly equal in concentration. The conjugated salts of their 7-alpha-dehydroxylated derivatives, deoxycholic acid and lithocholic acid, are also found, with derivatives of cholic, chenodeoxycholic and deoxycholic acids accounting for over 90% of human biliary bile acids.

The term “bile acid source” refers to a material which is capable of supplying a bile acid in the event that the material is in use. For example, the bile acid source may be a bile acid per se, a salt thereof, a conjugate thereof, a derivative thereof, or any mixture thereof. The term a derivative of a bile acid means a material which can transformed into a bile acid. For example, the derivative of a bile acid may be a conjugate of bile acid.

The term “agonist” is an agent that binds to a receptor and activates the receptor to produce a biological response, wherein the receptor is typically a member of a signaling pathway. Herein, a TGR5-GRK-β-arrestin-Src agonist is an agent that binds a receptor member of the TGR5-GRK-β-arrestin-Src pathway and activates the pathway to produce an enhanced immune response, particularly innate immune response. The term “antagonist” has opposite meaning against the “agonist”. An antagonist blocks or dampens a biological response by binding to and blocking a receptor rather than activating the receptor like an agonist. Binding of the antagonist to its receptor will disrupt the interaction of an receptor with its agonist and inhibit the function of the receptor.

Although it may be not true, in a particular embodiment, if it is known in the art that a compound functions as the agonist or antagonist of the present disclosure, the compound can be excluded from the definition of the agonist/antagonist in this particular context. For example, the TGR5-GRK-β-arrestin-Src agonist is an agent which is not known in the art to function as the TGR5-GRK-β-arrestin-Src agonist, while the TGR5-GRK-β-arrestin-Src antagonist is an agent which is not known in the art to function as the TGR5-GRK-β-arrestin-Src antagonist.

The term “pathway” or “signaling pathway” describes a group of molecules in a cell that work together to control one or more cell functions, such as innate immune response. After the first molecule in a pathway receives a signal, it activates another molecule. This process is repeated until the last molecule is activated and the cell function is carried out. For example, the TGR5-GRK-β-arrestin-Src pathway has essential members including TGR5, GRK, β-arrestin and Src, and enhance innate immune response when the pathway is activated. In a certain circumstance, blocking of an innate immunity pathway like TGR5-GRK-β-arrestin-Src may lead to health disorder such as cancer, and drugs can being developed to enhance the pathway, and these drugs may help block cancer cell growth and kill cancer cells.

### Examples

### Experimental Procedures

Reagents, Antibodies, Viruses and Cells

Kinase inhibitors BMS-345541, SAR-20347 and MRT67307 (MCE); INT-777 (MCE); LCA, CDCA, DCA and CA (Sigma); ISD45 and HSV120 (Sangon), poly(I:C) and 2′3′-cGAMP (Invivogen); digitonin (Sigma); lipofectamine 2000 (Invitrogen); IFN-γ (Peptech); BA assay kit (Sigma); ELISA kit for murine IFN-β and IFN-α (PBL); mouse monoclonal antibodies against HA (Covance), Flag and β-actin (Sigma); rabbit polyclonal antibodies against SRC, p-SRC(Y416), β-arrestin1, β-arrestin2, GRK6, MITA, p-p65, p-IKKα/β and IKKα/β and p-IRF3(S396) (CST), TBK1 and p-TBK1(S172) (Abcam), IRF3 and p65 (Santa Cruz Biotechnology) were purchased from the indicated manufacturers. Mouse antisera against murine TGR5, RIG-I, IRF3 and VISA were raised using the respective recombinant proteins as antigens. SeV, EMCV, HSV-1 and VSV-GFP were previously described (Hu et al, 2016 and 2017). HEK293 cells and HFFs were obtained from ATCC. HEK293T cells were originally provided by Dr. Gary Johnson (National Jewish Health). Primary Tgr5^{+/+} and Tgr5^{-/-} BMDMs, BMDCs and MLFs were prepared as described (Hu et al., 2015).

Constructs

Flag- and HA-tagged SRC, HA-tagged RIG-I, VISA, cGAS, MITA, TBK1, IRF3 or their mutants, CRISPR-Cas9 gRNA plasmids for IRF3, p65, VISA, MITA, β-arrestin1/2 and SRC, pSuper.Retro-shRNA plasmids (Oligoengine) for CYP7A1, CYP7B1, OATP, CYP27A1, HSD3B7, AKR1D1, AMACR, ACOX1, ACOX2, HSD17B4, EHHADH, SCP2, GNAI1, GNAI2, GNAI3, GNAS, ARRB1, ARRB2, GRK2, GRK3, GRK4, GRK5, GRK6 and SRC were constructed by standard molecular biology technique.

Tgr5 Knockout Mice

Tgr5 knockout mice were kindly provided by Dr. Di Wang (Zhejiang University, China). All animal experiments were performed in accordance with the Wuhan University animal care and use committee guidelines.

Transfection

HEK293 and 293T cells were transfected by standard calcium phosphate precipitation method. BMDMs were transfected by lipofectamine 2000 according to procedures recommended by the manufacturer.

Measurement of Intracellular BAs by Mass Spectrometry

BAs analysis be MS has been described (Zhu et al., 2015). Briefly, cell samples were thawed on the ice, then 1 mL methanol was added to the samples. After vortexing for 5 min, cells were crushed by ultrasonic cell crusher for another 5 min, followed by centrifugation at 13680 g at 4° C. for 5 min before the supernatants were collected. This procedure was repeated twice. The combined supernatant was dried under nitrogen gas and reconstituted in 100 μL acetonitrile followed by addition of 20 μL 2-chloro-1-methylpyridinium iodide (20 μmol/mL), 40 μL triethylamine (20 μmol/mL) and 40 μL d4-2-dimethylaminoethylamine (20 μmol/mL) in sequence (Zhu et al., 2015). The reaction solution was vortexed at 40° C. for 1 hour and then evaporated under nitrogen gas. Standard solutions (CA, LCA, CDCA, DCA) after chemical labeling with 2-dimethylaminoethylamine were used as internal standards. 2 ng/mL ISs were added to the samples followed by dissolved in 100 μL 20% acetonitrile (v/v) and subjected for LC-ESI-MS/MS system consisting of a Shimadzu MS-8050 mass spectrometer (Tokyo, Japan) with a Shimadzu LC-20AD HPLC system (Tokyo, Japan). The separation was performed on an Acquity UPLC BEH C18 column (2.1×100 mm, 1.7 μm, Waters) at 40° C. The mobile phase consisted of (A) ACN and (B) Formic acid in water (0.1%, v/v). A gradient of 0-2 min 80% B, 2-4 min 80% to 75% B, 4-8 min 75% to 72%, 8-11 min 72% to 70%, 11-12 min 70% to 40% B, 12-14 min 40% to 20% B, 14-15 min 20% to 10% B and 15-16 min 10% to 80% B was used. The flow rate of mobile phase was set as 0.4 mL/min. Multiple reaction monitoring (MRM) of [M+H]+ and the appropriate product ions were chosen to quantify BAs. Zhu, Q. F., Hao, Y H., Liu, M. Z., Yue, J., Ni, J., Yuan, B. F., Feng, Y Q., Journal of Chromatography A, 1410 (2015) 154-163

qPCR

Total RNA was isolated for qPCR analysis to measure mRNA levels of the indicated genes. Data shown are the relative abundance of the indicated mRNA normalized to that of GAPDH. qPCR was performed using the following primers:

Murine Ifnb1:
(SEQ ID NO: 1)
Forward-TCCTGCTGTGCTTCTCCACCACA;
(SEQ ID NO: 2)
Reverse-AAGTCCGCCCTGTAGGTGAGGTT.
Murine Isg56:
(SEQ ID NO: 3)
Forward-ACAGCAACCATGGGAGAGAATGCTG;
(SEQ ID NO: 4)
Reverse-ACGTAGGCCAGGAGGTTGTGCAT.
Murine Cxcl10:
(SEQ ID NO: 5)
Forward-ATCATCCCTGCGAGCCTATCCT;
(SEQ ID NO: 6)
Reverse-GACCTTTTTTGGCTAAACGCTTTC.
Murine Ifnb1:
(SEQ ID NO: 7)
Forward-TCCTGCTGTGCTTCTCCACCACA;
(SEQ ID NO: 8)
Reverse-AAGTCCGCCCTGTAGGTGAGGTT.
Murine Isg56:
(SEQ ID NO: 9)
Forward-ACAGCAACCATGGGAGAGAATGCTG;
(SEQ ID NO: 10)
Reverse-ACGTAGGCCAGGAGGTTGTGCAT.
Murine Cxcl10:
(SEQ ID NO: 11)
Forward-ATCATCCCTGCGAGCCTATCCT;
(SEQ ID NO: 12)
Reverse-GACCTTTTTTGGCTAAACGCTTTC.
Murine Gapdh:
(SEQ ID NO: 13)
Forward-ACGGCCGCATCTTCTTGTGCA;
(SEQ ID NO: 14)
Reverse-ACGGCCAAATCCGTTCACACC.

Coimmunoprecipitation and Immunoblot Analysis

Cells were lysed with RIPA buffer plus complete protease inhibitors and 20 mM NEM, and lysates were sonicated for 1 min. The lysates were centrifuged at 14000 rpm for 20 min at 4° C. The supernatants were incubated with respective antibodies at 4° C. overnight before protein G beads were added for 2 h. The beads were washed with cold PBS plus 0.5 M NaCl for three times followed by an additional wash with PBS. Proteins were separated by 8% SDS-PAGE, followed by immunoblot analysis with the indicated antibodies.

Plaque Assays

To measure HSV-1 replication in mouse brains, snap-frozen brains were weighed and homogenized for 3 times (each 5 seconds) in MEM medium. After homogenization, the brain suspensions were centrifuged at 1620 g for 30 minutes, and the supernatants were used for plaque assays on monolayers of Vero cells seeded in 24-well plates.

To measure HSV-1 replication in cells, cells were infected with HSV-1 (MOI=0.01) for the indicated times and then supernatants were collected for plaque assays on monolayers of Vero cells seeded in 24-well plates. The cells were infected by incubation for 1 hour at 37° C. with serial dilutions of brain suspensions. After 1 hour infection, 2% methylcellulose was overlaid, and the plates were incubated for about 48 hours. The overlay was removed and cells were fixed with 4% paraformaldehyde for 15 minutes and stained with 1% crystal violet for 30 minutes before plaque counting.

Statistics

For mouse experiments, no specific blinding method was used, but mice in each sample group were selected randomly. The sample size (n) of each experimental group is described in each corresponding figure legend. GraphPad Prism software was used for all statistical analyses. Quantitative data displayed as histograms are expressed as means±s.d. (represented as error bars). Data were analyzed using a Student's unpaired t-test and Log-rank (Mantel-Cox) test. The number of asterisks represents the degree of significance with respect to P values. Statistical significance was set at a P<0.05.

### Experimental Results

1. Viral Infection Induces Expression of BA Transporter and Synthesis Enzymes Via the Immediate Early NF-κB Activation

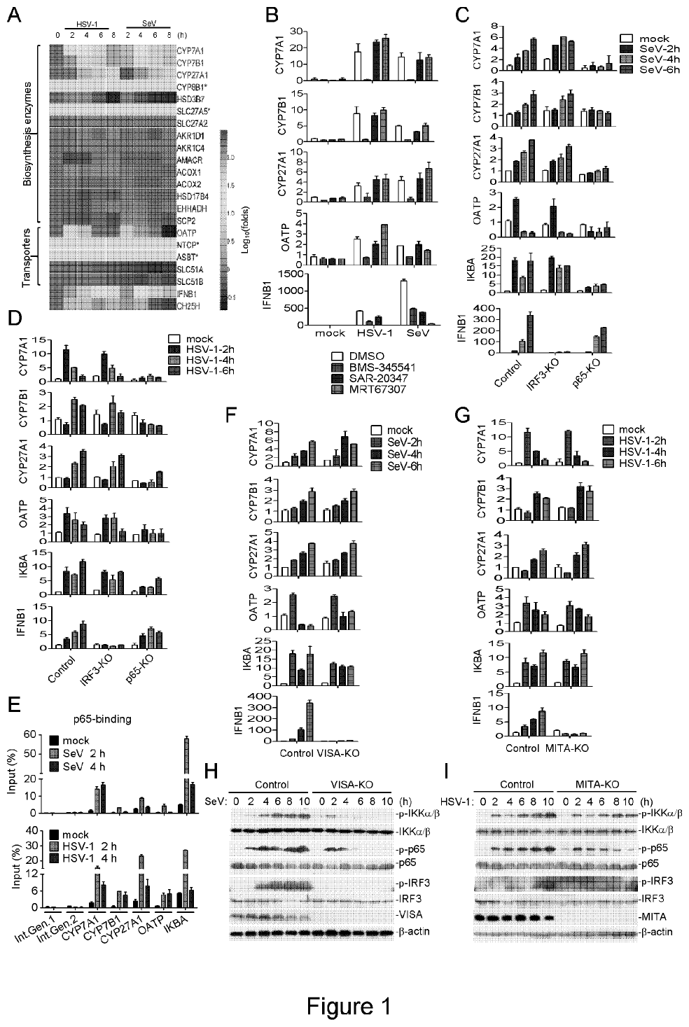

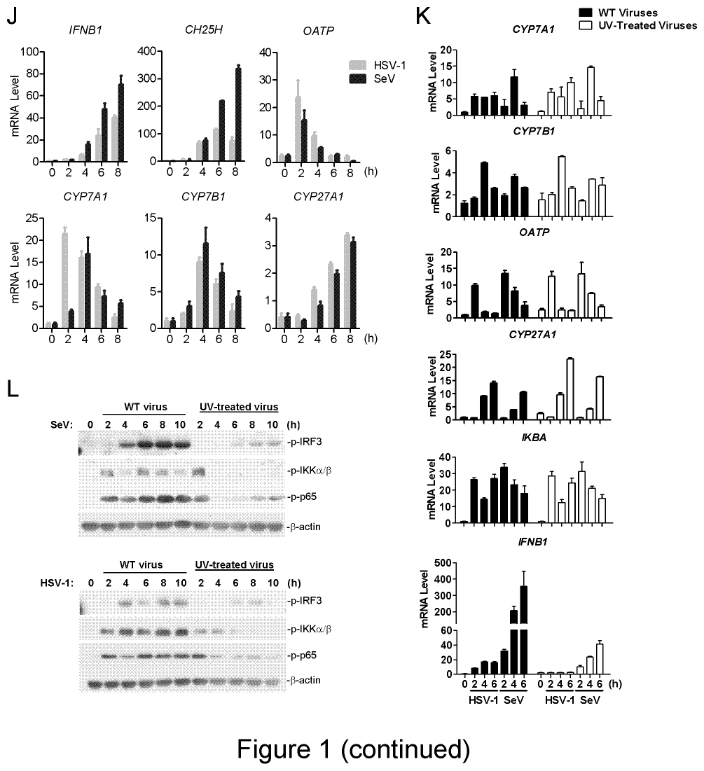

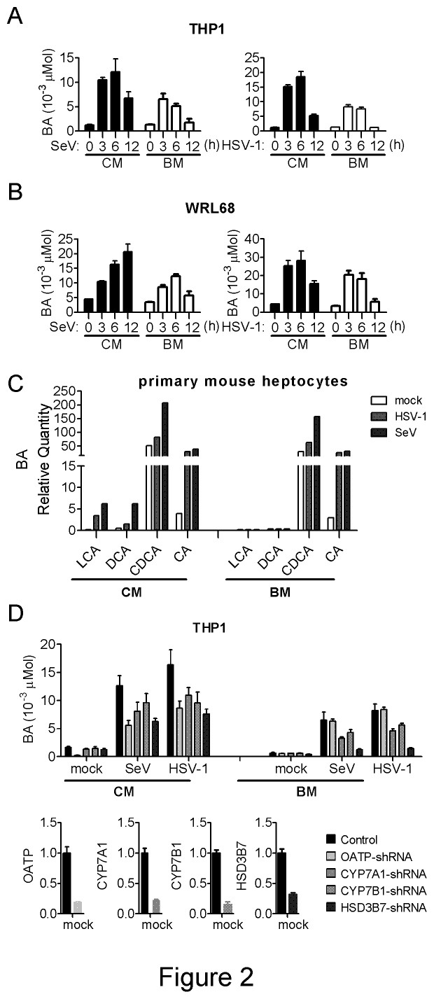

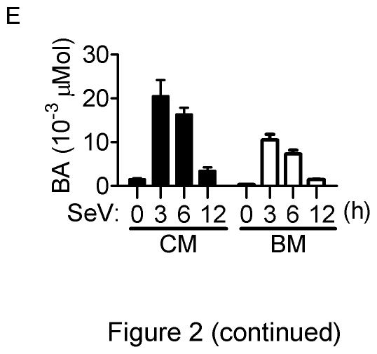

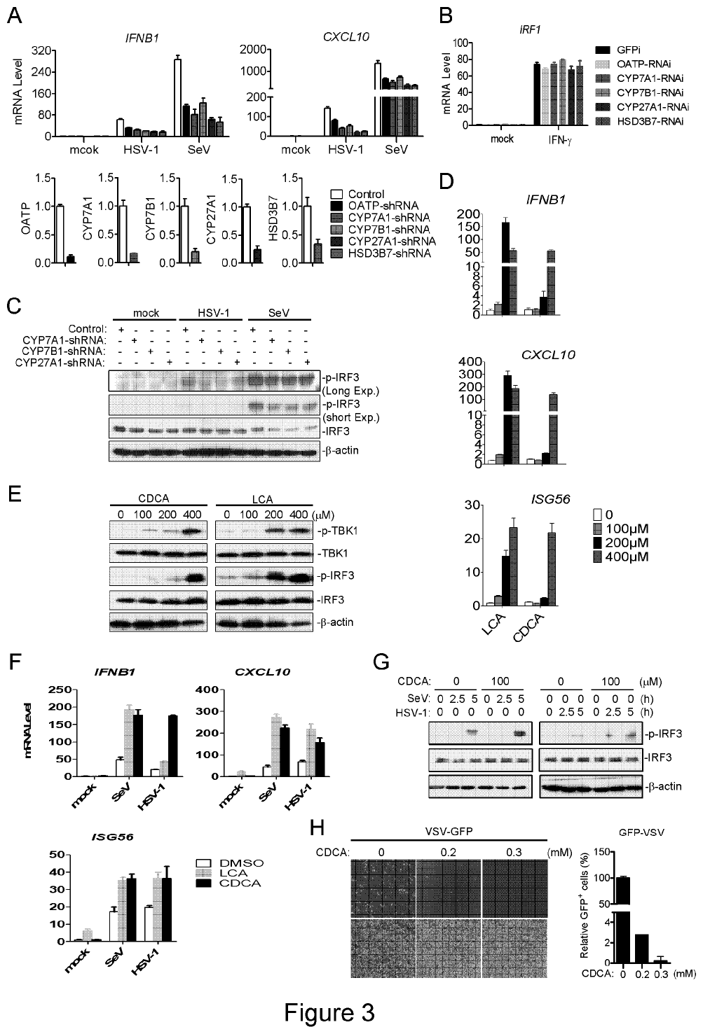

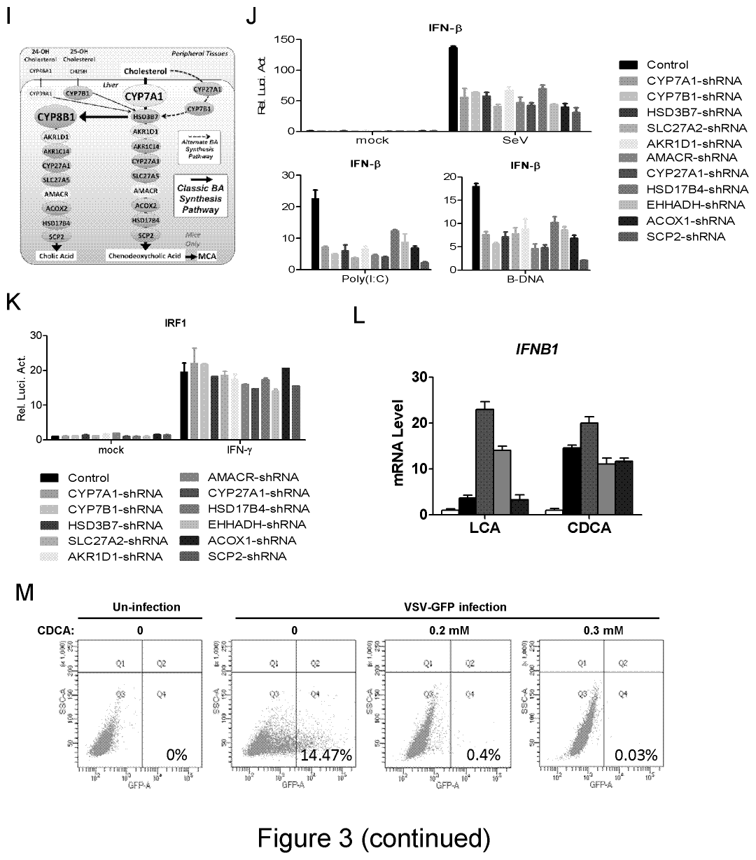

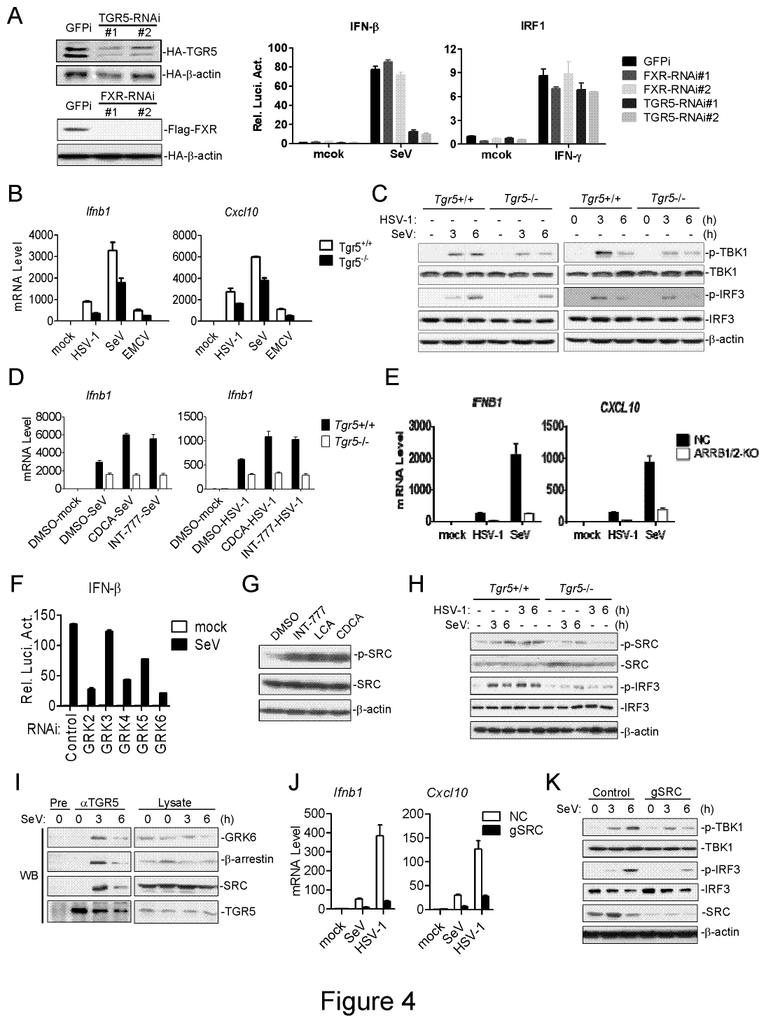

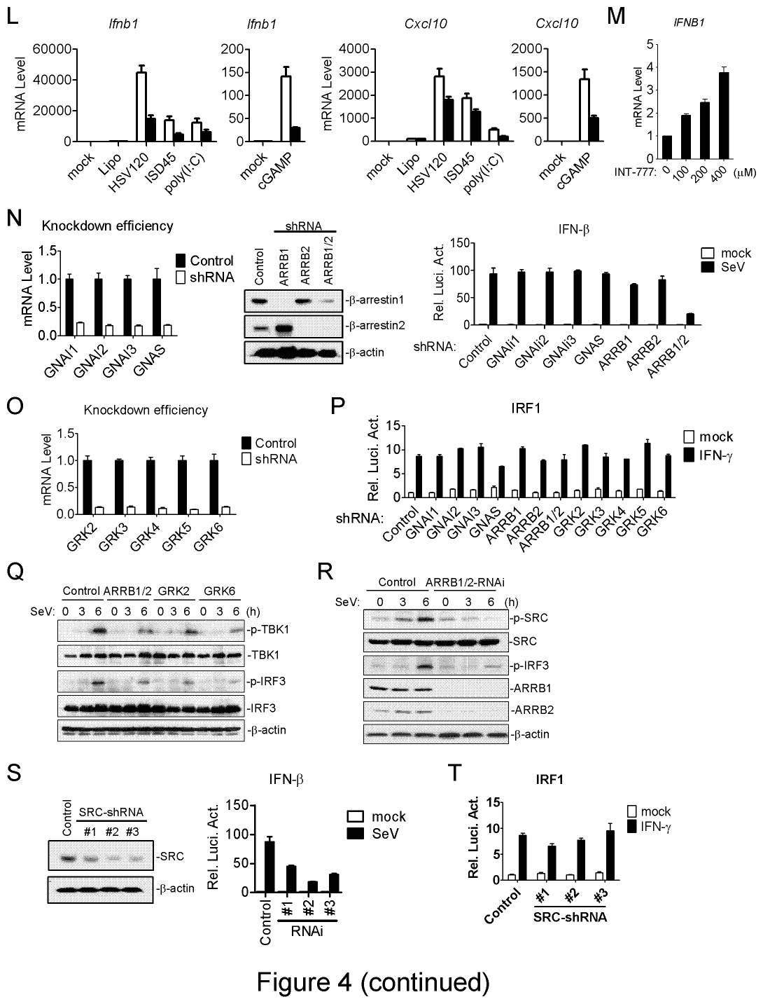

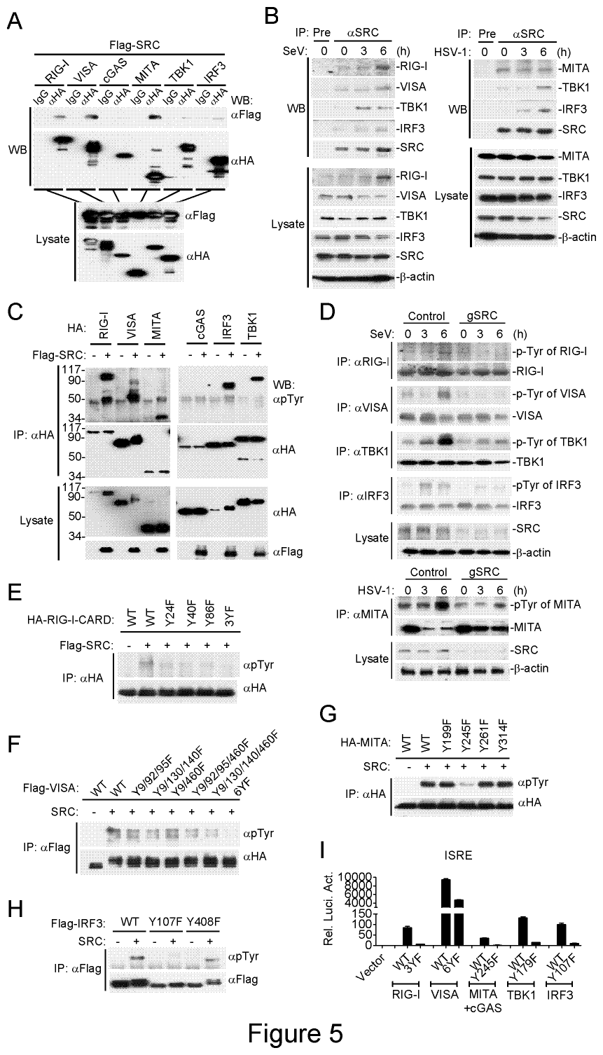

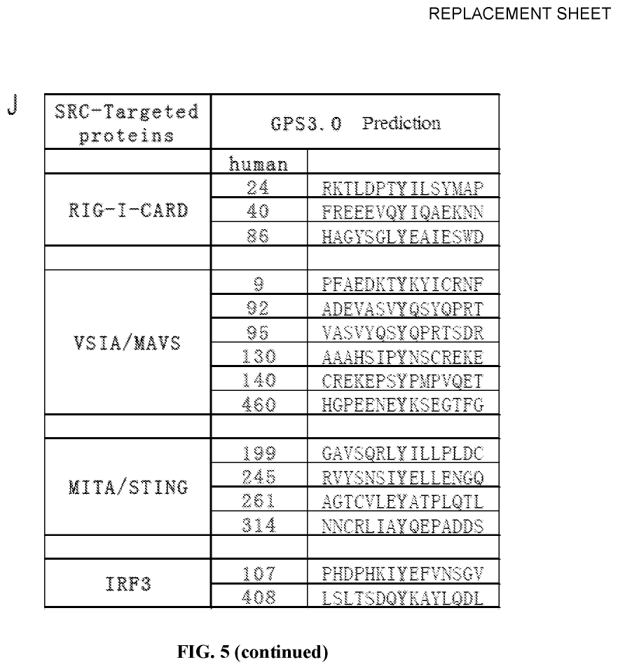

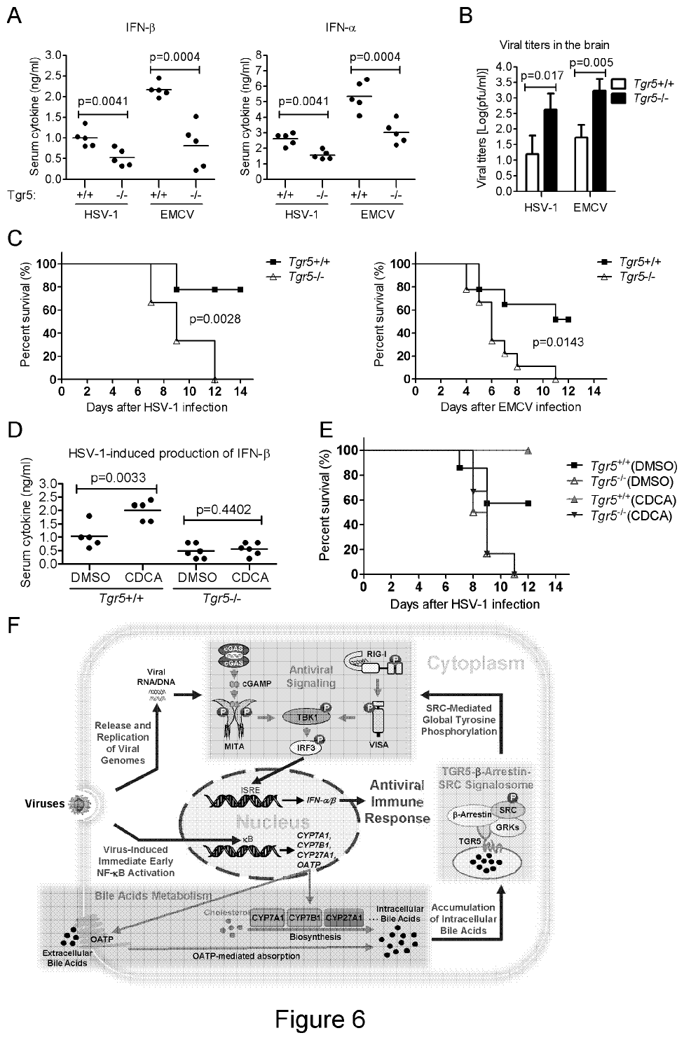

To investigate the relationship between BA metabolism and innate antiviral immunity, the inventors firstly examined expression of genes involved in BA biosynthesis and absorption in human monocyte THP1 cells following viral infection. Surprisingly, both the DNA virus herpes simplex virus 1 (HSV-1) and the RNA virus Sendai virus (SeV) specifically induced transcription of several critical rate-limiting enzymes involved in bile acid biosynthesis including CYP7A1, CYP7B1 and CYP27A1, as well as the plasma membrane-located bile acid transporter OATP (FIGS. 1A & 1J). In these experiments, HSV-1 and SeV also induced transcription of the classic antiviral gene IFNB1, but did not induce transcription of other non-rate-limiting enzymes including HSD3B7, SLC27A5, AKR1D1, AKR1C4, AMACR, ACOX1/2, HSD17B4, EHHADH and SCP2 or other transporters including SLC51A and SLC51B which mainly forms a heterodimer that acts as the intestinal basolateral transporter responsible for bile acid export from enterocytes into portal blood. (FIG. 1A). In addition, the expression of several liver- and enterohepatic tissue-restricted proteins including CYP8B1, SLC27A5, NTCP and ASTB were hardly detected in THP1 cells (FIG. 1A).

Viral infection activates the kinases IKKα/β and TBK1, which activates NF-κB and IRF3 respectively, leading to induction of type I interferons (IFNs) and proinflammatory cytokines. Type I IFNs further induces downstream antiviral genes via JAK-STAT pathways. To investigate how viral infection induces BA transporter and biosynthesis enzymes (TBEs), the inventors examined the effects of the IKKα/β inhibitor BMS-345541, the TBK1 inhibitor MRT67307, and the JAKs inhibitor SAR-20347 on virus-triggered transcription of BA TBE genes. The inventors found that BMS-345541 but not MRT67307 or SAR-20347 abolished HSV-1- and SeV-induced transcription of CYP7A1, CYP7B1, CYP27A1 and OATP genes in THP1 cells (FIG. 1). In the same experiments, all of the three inhibitors impaired virus-induced transcription of IFNB1 gene (FIG. 1B). These results suggest that virus-triggered NF-ΛB activation is essential for induction of BA TBE genes. Consistently, CRISPER/Cas9-mediated knockout of the NF-κB transactivator p65 but not IRF3 dramatically inhibited SeV- and HSV-1-induced transcription of BA TBE genes as well as the well-known NF-κB-targeted gene IKBA (FIG. 1C&D). CHIP assays indicated that viral infection induced the binding of p65 to the promoters of the BA TBE and IKBA genes but not to intergenic regions of chromatins (FIG. 1E). These results suggest that viral infection induces transcription of several key genes involved in bile acid biosynthesis and absorption in a NF-κB-dependent process.

The present experiments indicated that deficiency of VISA/MAVS or MITA/STING, which are essential adaptors in viral RNA- and DNA-triggered NF-κB activation pathways respectively, impaired SeV- and HSV-1-induced transcription of IFNB1 gene respectively, but had no marked effects on virus-induced transcription of BA TBE (CYP7A1, CYP7B1, CYP27A1, OAT) and IKBA genes (FIG. 1F&G). Interestingly, biochemical analysis showed that while VISA/MAVS- or MITA/STING-deficiency abolished SeV- and HSV-1-induced phosphorylation of IRF3 respectively, their deficiencies only impaired the phosphorylation of IKKα/β and p65, which are hallmarks of NF-κB activation, at the late phase (4-10 h) but not the immediate early phase (2 h) after viral infection (FIG. 1H&I). These results suggest that the first wave and VISA- or MITA-independent activation of NF-κB following viral infection drives the transcription of BA TBE genes. Consistently, UV treatment of viruses, which impairs viral replication but not entry into the cell, impaired HSV-1- or SeV-induced transcription of IFNB1 gene but did not affect virus-induced transcription of the BA TBE genes (FIG. 1K). In addition, UV-treated viruses induced normal phosphorylation of IKKα/β and p65 at the immediate early phase (2 h) of viral infection but failed to induce their phosphorylation at the late phase (4-10 h) (FIG. 1L). In the same experiments, phosphorylation of IRF3 was induced at 4 h post SeV or HSV-1 infection in wild-type cells, which was abolished in VISA- or MITA-deficient cells respectively, or by UV-treatment of the viruses (FIG. 1H&I, FIG. 1L). These results suggest that IRF3 activation occurs after the first wave of VISA- and MITA-independent NF-κB activation, and requires viral replication and VISA- or MITA-dependent signaling respectively.

2. Viral Infection Induces Accumulation of Intracellular BAs

The inventors unexpectedly found that both HSV-1 and SeV infection caused dramatic increase of intracellular BA levels in human monocytic THP1, hepatic WRL68 and HEK293 cells that is cultured in either FBS-containing complete medium (CM) or FBS-free basic medium (BM) (FIG. 2A&B, FIG. 2E). Interestingly, the increase of BA levels after viral infection was ˜40% lower in cells cultured in BM in comparison to CM (FIG. 2A&B, FIG. 2E). Since CM contains BAs but BM does not, these results indicate that both de novo biosynthesis and extracellular to intracellular transportation contribute to virus-induced accumulation of intracellular BAs. The present results also showed that the BA levels were rapidly increased at the early phase (3-6 h) and then decreased at the late phase (12 h) after viral infection, consistent with critical roles of BAs in regulating innate antiviral response (see below). The only exception was that BA levels consistently increased without decrease by 12 h after SeV infection in hepatic WRL68 cultured in CM (FIG. 2B). It is possible that hepatic cells respond longer for RNA virus-induced uptake of extracellular BAs.

BAs include various primary and secondary molecules. The inventors further determined what BAs were accumulated after viral infection by mass spectrometry (MS). The inventors found that only the primary bile acids CDCA and CA but not the secondary bile acid LCA or DCA was dramatically increased in primary mouse hepatocytes cultured with BM after HSV-1 or SeV infection for 6 hrs, suggesting that only the primary BAs are biosynthesized after viral infection. However, all examined BAs, including CDCA, CA, LCA and DCA, were increased in primary mouse hepatocytes cultured in CM after HSV-1 or SeV infection for 6 hrs (FIG. 2C), suggesting that viral infection induces uptake from the medium of both the primary bile acids CDCA and CA and the secondary bile acids LCA and DCA.

Next the inventors investigated the contribution of the BA transporter and biosynthesis enzymes to virus-induced intracellular accumulation of BAs. The inventors found that HSV-1- and SeV-induced increase of BA levels in THP1 cells cultured in CM was significantly inhibited by shRNA-mediated knockdown of BA biosynthesis enzyme CYP7A1, CYP7B1, or HSD3B7 (a common downstream enzyme of CYP7A1 and CYP7B1), or the BA transporter OATP (FIG. 2D). When THP1 was cultured in BM, HSV-1- or SeV-induced increase of intracellular BA levels was significantly inhibited by knockdown of CYP7A1, CYP7B1 or HSD3B7 but not OATP (FIG. 2D). Taken together, these results suggest that viral infection-induced accumulation of intracellular BAs is caused by both CYP7A1- and CYP7B1-mediated de novo biosynthesis as well as OATP-mediated uptake from the medium.

3. BAs are Potent Inducers of Innate Immune Response

The inventors further investigated the significance of BAs in innate immunity, using innate anti-viral immune response as an example. The inventors surprisingly found that knockdown of OATP, CYP7A1, CYP7B1, CYP27A1 or HSD3B7 markedly inhibited HSV-1- or SeV-induced transcription of downstream antiviral IFNB1 and CXCL10 genes (FIG. 3A). As a control, knockdown of these BA transporter and biosynthesis enzymes did not affect IFN-γ-induced transcription of IRF1 gene (FIG. 3B). Consistently, virus-induced phosphorylation of IRF3 was markedly inhibited in CYP7A1-, CYP7B1- or CYP27A1-knock down cells (FIG. 3C). Reporter assays indicated that knockdown of all the examined enzymes involved in BA biosynthesis pathways markedly inhibited SeV- as well as transfected synthetic poly(I:C)- and B-DNA-induced activation of the IFN-β promoter (FIG. 3I&J), but had no marked effects on IFNγ-induced activation of the IRF1 promoter (FIG. 3K). These results suggest that the biosynthesis of BAs plays an important role in innate immune response.

The inventors next determined whether BAs directly regulate innate immune response. The inventors found that LCA and CDCA potently induced transcription of downstream innate immunity genes including IFNB1, CXCL10 and ISG56 (FIG. 3D). Consistently, LCA and CDCA also induced phosphorylation of TBK1 and IRF3, which are hallmarks of activation of these essential innate immune signaling components (FIG. 3E). Time-course experiments indicated that the BAs induced the transcription of IFNB1 gene as early as 2 h and peak at 4 hours post stimulation (FIG. 3L). Furthermore, exogenous BAs could also potentiate HSV-1- or SeV-induced transcription of IFNB1, CXCL10 and ISG56 genes (FIG. 3F), as well as phosphorylation of IRF3 (FIG. 3G). In addition, both fluorescence microscope experiments and flow cytometry analysis indicated that treatment with CDCA dramatically inhibited VSV-GFP replication (FIG. 3H&M). These results suggest that BAs are potent inducers of innate immune response.

4. BAs Promote Innate Immune Response Via a TGR5-GRK-β-Arrestin-SRC Axis

The inventors next investigated the mechanisms of BA-induced innate immune response. The inventors unexpectedly found that knockdown of TGR5 but not FXR dramatically inhibited SeV-induced activation of the IFN-β promoter, while knockdown of either TGR5 or FXR had no marked effects on IFN-γ-induced activation of the IRF1 promoter in reporter assays (FIG. 4A), suggesting that TGR5 but not FXR plays an important role in innate antiviral immune response. Consistently, TGR5-deficient mouse macrophages expressed lower levels of Ifnb1 and Cxcl10 mRNAs in response to infection with HSV-1, SeV or Encephalomyocarditis virus (EMCV), transfection with synthetic viral DNA mimics HSV120 (dsDNA (120-mers) representing the genomes of HSV-1) or ISD45 (IFN-stimulating DNA 45), poly(I:C), or cGAMP (FIG. 4B&4L). In addition, HSV-1- and SeV-induced phosphorylation of TBK1 and IRF3 was also inhibited in TGR5-deficient macrophages (FIG. 4C). Furthermore, CDCA and INT-777, a selective TGR5 agonist (FIG. 4M), potentiated HSV-1- and SeV-induced transcription of Ifnb1 in Tgr5^{+/+} but not Tgr5^{-/-} macrophages (FIG. 4D). These results suggest that the BA receptor TGR5 mediates innate immune response.

TGR5 is a member of G-protein coupled receptor (GPCR) family, which activates distinct downstream effector proteins including G proteins and β-arrestin1/2. The inventors found that knockdown of β-arrestin1 or β-arrestin2 simultaneously but not individually markedly inhibited SeV-induced transcription of activation of IFN-β in reporter assays (FIG. 4N). In the same experiments, knockdown of various subtypes of G proteins including several Ga (inhibitory G protein subunit a encoded by GNAI1, GNAI2 and GNAI3) and Gas (stimulatory G protein subunit a encoded by GNAS) didn't have any marked effects on SeV-induced activation of IFN-β (FIG. 4N). Consistently, Simultaneous knockout of ARRB1 (encoding for β-arrestin1) and ARRB2 (encoding for β-arrestin2) genes by the CRISPR/Cas9 method dramatically inhibited HSV-1- or SeV-induced transcription of IFNB1 and CXCL10 genes in THP-1 cell (FIG. 4E). These results suggest that β-arrestin1 and β-arrestin2 play redundant roles in innate antiviral immune response. It has been well established that GPCR-related kinases (GRKs) are required for recruitment of β-arrestins to activated GPCR (Premont et. al., 2007). The inventors found that knockdown of GRK2, GRK4, or GRK6 markedly inhibited SeV-induced activation of the IFN-β promoter, while knockdown of GRK3 or GRK5 had minimal effects (FIG. 4F&4O). As controls, knockdown of β-arrestins or GRKs had no marked effects on IFN-γ-induced activation of the IRF1 promoter in reporter assays (FIG. 4P). Further biochemical experiments indicated that SeV-induced phosphorylation of TBK1 and IRF3 were markedly inhibited in β-arrestin1/2-, GRK2- and GRK6-deficient cells (FIG. 4Q). These results suggest that the TGR5-GRK-β-arrestin axis mediates BA-induced innate immune response.

The inventors next determined whether intracellular BAs activate SRC via TGR5-GRK-β-arrestin axis in response to viral infection. Biochemical experiments indicated that BA and INT-777 treatments as well as viral infection induced phosphorylation of SRC at Y416, which is a hallmark for SRC activation (Cooper et al., 1993) (FIG. 4Q 4H&4R), and Tgr5-deficiency or knockdown of β-arrestin1/2 impaired SeV-induced phosphorylation of SRC and IRF3 (FIG. 4H). Endogenous coimmunoprecipitation experiments indicated that SeV infection induced the recruitment of β-arrestin, GRK6 and SRC to TGR5 at 3 h after viral infection (FIG. 4). Further experiments indicated that CRISPR/Cas9-mediated knockout of SRC dramatically inhibited SeV-induced transcription of Ifnb1 and Cxcl10 genes as well as phosphorylation of TBK1 and IRF3 (FIG. 4J&K). These results suggest that BA-triggered and TGR5-GRK-β-arrestin-mediated SRC activation is essential for efficient innate immune response.

5. SRC Mediates Tyrosine Phosphorylation and Activation of Multiple Key Components in Innate Immune Signaling Pathways

The inventors further investigated whether SRC regulates other components in innate antiviral immune signaling pathways. Transient transfection and coimmunoprecipitation experiments indicated that SRC strongly interacted with RIG-I, VISA and MITA, and weakly interacted with TBK1 and IRF3, but had no interaction with cGAS (FIG. 5A). Endogenous coimmunoprecipitation experiments indicated that the association of SRC with RIG-I, VISA/MAVS, TBK1 and IRF3 was increased after viral infection, while SRC was associated with MITA/STING constantly (FIG. 5B). In addition, overexpression of SRC caused tyrosine phosphorylation of RIG-I, VISA, MITA, TBK1 and IRF3, but not cGAS in (FIG. 5C), whereas SRC-deficiency impaired SeV- or HSV-1-induced tyrosine phosphorylation of endogenous RIG-I, VISA/MAVS, TBK1, IRF3 and MITA/STING (FIG. 5D). These results suggest that SRC mediates virus-induced tyrosine phosphorylation of multiple proteins in innate immune signaling pathways.

The inventors next investigated the functional significance of SRC-mediated tyrosine phosphorylation of the key components in innate antiviral immune signaling pathways. Multiple potential SRC-targeted tyrosine residues were identified for the CARD domain of RIG-I (which is responsible for signaling downstream activation), VISA, MITA and IRF3 by the GPS3.0 program (FIG. 5J). Biochemical analysis showed that simultaneous mutation of Y24, Y40 and Y86 to phenylalanine (F) (RIG-I-CARD-Y3F) completely abolished SRC-mediated tyrosine phosphorylation of RIG-I-CARD (FIG. 5E), indicating that the three residues are targeted by SRC. Simultaneous mutation of Y9, Y92, Y95, Y130, Y140 and Y460 of VISA to F (VISA-6YF) impaired SRC-mediated tyrosine phosphorylation (FIG. 5F), indicating that these residues are targeted by SRC. Mutation of Y245 of MITA to F (MITA-Y/F) abolished SRC-mediated tyrosine phosphorylation of MITA (FIG. 5G), indicating that SRC catalyzes MITA phosphorylation at Y245. Mutation of Y107 of IRF3 to F (IRF3-Y/F) impaired SRC-mediated tyrosine phosphorylation of IRF3 (FIG. 5H), indicating that SRC catalyzes IRF3 phosphorylation at Y107. Reporter assays indicted that these mutants lost the abilities to activate ISRE in comparison to their wild-type counterparts in reporter assays (FIG. 5I). These results suggest that SRC-mediated tyrosine phosphorylation of the key components in the innate antiviral immune response pathways is important for their activation.

6. The BA-TGR5 Pathway is Important for Host Defense Against Viral Infection In Vivo

Finally, the inventors determined the importance of the BA-TGR5 axis in innate antiviral immunity in vivo. ELISA experiments indicated that HSV-1- and EMCV-induced production of serum cytokines including IFN-β and IFN-α was significantly inhibited in Tgr5^{-/-} mice compared with their wild-type littermates (FIG. 6A). Consistently, much higher viral loads were detected in the brains of Tgr5^{-/-} mice 3 days after infection with HSV-1 or EMCV (FIG. 6B). Furthermore, Tgr5^{-/-} mice were significantly more susceptible to HSV-1- and EMCV-induced death (FIG. 6C). These results suggest that TGR5 is required for efficient host defense against viral infection in vivo. Furthermore, CDCA treatment potentiated HSV-1-induced production of IFN-β in wild-type but not TGR5-deficient mice (FIG. 6D), as well as significantly increased the survival rate of wild-type but not TGR5-deficient mice infected with HSV-1 (FIG. 6E), which further confirms an important role of BA-TGR5 pathway in innate immune response.

Therefore, the inventors put up a work model for elucidating the delicate regulatory relationships of BA metabolism and antiviral innate immunity (FIG. 6F). Briefly, viral infection triggers an immediate early NF-κB activation which results in a rapid induction of CYP7A1, CYP7B1, CYP27A1 and OATP, several critical proteins involved in BA biosynthesis and absorption, leading to accumulation of intracellular BAs via both absorption and biosynthesis. Then, accumulated BAs activate TGR5-GRK-β-arrestin-SRC pathway, followed by SRC-mediated global tyrosine phosphorylation of multiple proteins in antiviral immune signaling, including RIG-I, VSIA/MAVS, MITA/STING, TBK1 and IRF3, which is critical for their activation and enables the antiviral innate immune response.

### Discussion

Decoding the intrinsic principles of inter-regulation between cell metabolism and immunity attracts extensive attention in recent years, since it might bring some new directions for treatment of both metabolic and immune diseases. The present findings that viral infection-triggered accumulation of intracellular BAs via the immediate early NF-κB activation enables antiviral innate immune response by TGR5-GRK-β-arrestin-SRC pathway establish a delicate circuit between BA metabolism and antiviral innate immunity, which probably benefits in searching new approaches for treatment of metabolic and immune diseases.

Biosynthesis of BAs is believed to be exclusively restricted to the liver due to the extremely low levels of the rate-limiting enzyme CYP7A1 involved in the classical BA biosynthesis pathway in many extrahepatic tissues. Though the rate-limiting enzymes CYP27A1 and CYP7B1 in an alternative (acidic) pathway of bile acid biosynthesis are also expressed in macrophage and other tissues in addition to the liver, no strong evidences have demonstrated whether BAs could also be biosynthesized via this pathway in extrahepatic tissues. In this study, the inventors demonstrate that biosynthesis of BAs is activated in many types of cells upon viral infection owing to the induction of several rate-limiting enzymes including CYP7A1, CYP7B1 and CYP27A1 involved in BA biosynthesis, which provides a new perspective on basic knowledge of BA metabolism. Furthermore, ever since the identification of BAs as hormones several decades of years ago, intensive studies have revealed their hormonal functions, including direct metabolic actions on glucose and lipid metabolism in the liver and the intestines through the nuclear bile acid receptor FXR, as well as regulation of metabolic, endocrine and neurological processes of via another plasma membrane-bounded bile acid receptor TGR5. Without exception, all these reported functions are executed by ectocytic BAs biosynthesized and secreted from the liver or absorbed from the intestine. For the first time, the present findings that viral infection-induced accumulation of intracellular BAs via both biosynthesis and absorption facilitates antiviral innate immune response through activating intracellular TGR5-GRK-β-arrestin-SRC pathway reveal a universal and intrinsic cellular function of BA metabolism in many cells.

In the present study, the inventors strongly demonstrate that viral infection-triggered expression of the TBE genes (CYP7A1, CYP7B1, CYP27A1 and OATP) is dependent of the immediate early NF-κB activation with a series of biochemical and genetic experiments and analysis. During viral infection, several mechanisms are probably involved in NF-κB activation, such as virus-cell membrane fusion, antiviral innate immune response, viral replication and expression of some virus-specific proteins. According to the present results, the inventors propose that viral infection-induced expression of these TBE genes is probably dependent of virus-cell membrane fusion-triggered signaling cascade, which is prior to the innate immune response and viral replication. This contributes to and is consistent with the present further finding that induction of these TBE genes is critical for activation of the antiviral innate immune response.

By exploring the function of biosynthesis and absorption of BAs in virus-triggered signaling, the inventors demonstrate that accumulation of intracellular BAs is required for the optimal antiviral innate immune response. Further investigation revealed TGR5 but not FXR is the main receptor responsible for BAs-mediated potentiation of antiviral innate immune response. As a GPCR, TGR5 is mainly expressed on plasma membrane and usually mediates extracellular BAs-triggered signaling for regulation of many intracellular biological processes, which might partially account for type I IFN induction by exogenous BAs treatment. However, in the present study, the inventors demonstrate that TGR5 is also able to be activated by accumulation of intracellular bile acids, implying that TGR5 is probably distributed on intracellular organelles in addition to the plasma membrane, which is consistent with previous reports that TGR5 is also distributed on the endosome and nucleic membrane (PMID: 23578785, PMID: 19582812). The precise intracellular location of TGR5 and whether TGR5 is translocated from plasma membrane and intracellular organelles during viral infection need further study.

Though BAs-TGR5-cAMP-PKA signaling pathway has been well-established to be involved in the regulation of various biological processes, the present investigation indicated that accumulation of intracellular BAs activate TGR5-GRK-β-arrestin-SRC pathway for potentiation of antiviral innate immune response, since deficiency of any proteins in this pathway markedly impaired virus-triggered production of type I IFNs. Furthermore, endogenous associations of β-arrestin, GRK6 and SRC with TGR5 were detected following viral infection. In the present experiments, the inventors noticed that TGR5-deficiency completely blocked HSV-1-induced but only partially impaired SeV-induced phosphorylation of SRC at Y416, which implies that HSV-1-induced activation of SRC is completely dependent of BA-TGR5 pathway while SeV-induced activation of SRC is only partially dependent of BA-TGR5 pathway. With the results that mutation of SRC-targeted phosphorylation sites of multiple proteins in antiviral signaling dramatically impaired their activation of IFN-β, the inventors suggest that pan-tyrosine phosphorylation by SRC of the antiviral pathways is critical for their activation, which is consistent with the results that SRC-deficiency markedly impaired viruses-triggered induction of IFNB1 and other antiviral genes, though the detailed mechanisms for the regulation of the multiple proteins in antiviral signaling by SRC-mediated pan-tyrosine phosphorylation need further investigation in the future.

During the present investigation, the inventors notice that phosphorylation of SRC at Y416 is easily detected in both Tgr5^{+/+} and Tgr5^{-/-} cells either before and after viral infection, which might account for the rest activation of antiviral signaling and production of type I IFNs in TGR5-deficiency cells. Since SRC is able to be activated by many different classes of cellular receptors including immune response receptors, integrins and other adhesion receptors, receptor protein tyrosine kinases, G protein-coupled receptors as well as cytokine receptors (Cooper et al., 1993; Schlessinger, 2000), some other mechanism(s) might also exist in activation of SRC before or after viral infection, in addition to certain unavoidable factors for uncontrollable and nonspecific SRC activation in FBS-cultured cells of in vitro experiments. Whatever it takes, the present in vivo experiments showed that BA-TGR5 pathway plays an important role in innate immunity including host defense against infection, since TGR5-deficiency mice were much more susceptible to virus-induced death. Furthermore, BA treatment significantly increased the serum cytokine production and survival rates of the wild-type but not TGR5-deficiency mice, which not only further confirmed the important role of BA-TGR5 pathway in antiviral innate immune response, but also suggest that BAs are applicable and potent antiviral agents.

### REFERENCE

1. O. Takeuchi, S. Akira, Pattern recognition receptors and inflammation. Cell 140, 805-820 (2010); published online EpubMar 19 (10.1016/j.cell.2010.01.022).
2. J. T. Crowl, E. E. Gray, K. Pestal, H. E. Volkman, D. B. Stetson, Intracellular Nucleic Acid Detection in Autoimmunity. Annual review of immunology 35, 313-336 (2017); published online EpubApr 26 (10.1146/annurev-immunol-051116-052331).
3. R. B. Seth, L. Sun, C. K. Ea, Z. J. Chen, Identification and characterization of MAVS, a mitochondrial antiviral signaling protein that activates NF-kappaB and IRF 3. Cell 122, 669-682 (2005); published online EpubSep 9 (10.1016/j.cell.2005.08.012).
4. T. Kawai, K. Takahashi, S. Sato, C. Coban, H. Kumar, H. Kato, K. J. Ishii, O. Takeuchi, S. Akira, IPS-1, an adaptor triggering RIG-1- and Mda5-mediated type I interferon induction. Nat Immunol 6, 981-988 (2005); published online EpubOct (ni1243 [pii]10.1038/ni1243).
5. L. G. Xu, Y. Y. Wang, K. J. Han, L. Y. Li, Z. Zhai, H. B. Shu, VISA is an adapter protein required for virus-triggered IFN-beta signaling. Mol Cell 19, 727-740 (2005); published online EpubSep 16 (10.1016/j.molcel.2005.08.014).
6. E. Meylan, J. Curran, K. Hofmann, D. Moradpour, M. Binder, R. Bartenschlager, J. Tschopp, Cardif is an adaptor protein in the RIG-1 antiviral pathway and is targeted by hepatitis C virus. Nature 437, 1167-1172 (2005); published online EpubOct 20 (10.1038/nature04193).
7. L. Sun, J. Wu, F. Du, X. Chen, Z. J. Chen, Cyclic GMP-AMP synthase is a cytosolic DNA sensor that activates the type I interferon pathway. Science 339, 786-791 (2013); published online EpubFeb 15 (10.1126/science.1232458).
8. P. Gao, M. Ascano, Y. Wu, W. Barchet, B. L. Gaffney, T. Zillinger, A. A. Serganov, Y. Liu, R. A. Jones, G. Hartmann, T. Tuschl, D. J. Patel, Cyclic [G(2′,5′)pA(3′,5′)p] is the metazoan second messenger produced by DNA-activated cyclic GMP-AMP synthase. Cell 153, 1094-1107 (2013); published online EpubMay 23 (10.1016/j.cell.2013.04.046).
9. B. Zhong, Y. Yang, S. Li, Y. Y. Wang, Y. Li, F. Diao, C. Lei, X. He, L. Zhang, P. Tien, H. B. Shu, The adaptor protein MITA links virus-sensing receptors to IRF3 transcription factor activation. Immunity 29, 538-550 (2008); published online EpubOct 17 (10.1016/j.immuni.2008.09.003).
10. H. Ishikawa, Z. Ma, G. N. Barber, STING regulates intracellular DNA-mediated, type I interferon-dependent innate immunity. Nature 461, 788-792 (2009); published online EpubOct 8 (10.1038/nature08476).
11. X. Cai, Y. H. Chiu, Z. J. Chen, The cGAS-cGAMP-STING pathway of cytosolic DNA sensing and signaling. Mol Cell 54, 289-296 (2014); published online EpubApr 24 (10.1016/j.molcel.2014.03.040).
12. C. Chiang, M. U. Gack, Post-translational Control of Intracellular Pathogen Sensing Pathways. Trends in immunology 38, 39-52 (2017); published online EpubJan (10.1016/j.it.2016.10.008).
13. K. Ganeshan, A. Chawla, Metabolic regulation of immune responses. Annual review of immunology 32, 609-634 (2014) 10.1146/annurev-immunol-032713-120236).
14. B. Kelly, L. A. O'Neill, Metabolic reprogramming in macrophages and dendritic cells in innate immunity. Cell research 25, 771-784 (2015); published online EpubJul (10.1038/cr.2015.68).
15. D. W. Russell, Fifty years of advances in bile acid synthesis and metabolism. Journal of lipid research 50 Suppl, S120-125 (2009); published online EpubApr (10.1194/jlr.R800026-JLR200).
16. T. Q. de Aguiar Vallim, E. J. Tarling, P. A. Edwards, Pleiotropic roles of bile acids in metabolism. Cell metabolism 17, 657-669 (2013); published online EpubMay 7 (10.1016/j.cmet.2013.03.013).
17. Y. Kawamata, R. Fujii, M. Hosoya, M. Harada, H. Yoshida, M. Miwa, S. Fukusumi, Y. Habata, T. Itoh, Y. Shintani, S. Hinuma, Y. Fujisawa, M. Fujino, A G protein-coupled receptor responsive to bile acids. J Biol Chem 278, 9435-9440 (2003); published online EpubMar 14 (10.1074/jbc.M209706200).
18. T. Maruyama, Y. Miyamoto, T. Nakamura, Y. Tamai, H. Okada, E. Sugiyama, T. Nakamura, H. Itadani, K. Tanaka, Identification of membrane-type receptor for bile acids (M-BAR). Biochem Biophys Res Commun 298, 714-719 (2002); published online EpubNov 15 (
19. T. W. Pols, L. G. Noriega, M. Nomura, J. Auwerx, K. Schoonjans, The bile acid membrane receptor TGR5 as an emerging target in metabolism and inflammation. Journal of hepatology 54, 1263-1272 (2011); published online EpubJun (10.1016/j.jhep.2010.12.004).
20. C. Guo, S. Xie, Z. Chi, J. Zhang, Y. Liu, L. Zhang, M. Zheng, X. Zhang, D. Xia, Y. Ke, L. Lu, D. Wang, Bile Acids Control Inflammation and Metabolic Disorder through Inhibition of NLRP3 Inflammasome. Immunity 45, 802-816 (2016); published online EpubOct 18 (10.1016/j.immuni.2016.09.008).
21. R. T. Premont, R. R. Gainetdinov, Physiological roles of G protein-coupled receptor kinases and arrestins. Annual review of physiology 69, 511-534 (2007) 10.1146/annurev.physiol.69.022405.154731).
22. J. A. Cooper, B. Howell, The when and how of Src regulation. Cell 73, 1051-1054 (1993); published online EpubJun 18
23. J. Schlessinger, New roles for Src kinases in control of cell survival and angiogenesis. Cell 100, 293-296 (2000); published online EpubFeb 4 (

1. A method of enhancing immunity in a subject in need thereof, comprising administering a TGR5-GRK-β-arrestin-Src agonist to the subject.

2. The method of claim 1, wherein the immunity is an immunity against microbial infection, e.g., bacterial infection, viral infection, fungal infection.

3. The method of claim 2, wherein the viral infection is caused by DNA virus or RNA virus such as ssDNA virus, dsDNA virus, ssRNA virus or dsRNA virus, e.g. virus selected from the group consisting of herpes simplex virus (HSV) including HSV-1 and HSV-2, human cytomegalovirus (HCMV), Kaposi's sarcoma-associated herpes virus (KSHV), hepatitis B virus (HBV), hepatitis C virus (HCV), Zika Virus, EV71, influenza virus, Human Immunodeficiency Virus (HIV), EB virus, and human papillomavirus (HPV).

4. The method of claim 2, wherein the microbial infection induces a disease, which is for example selected from the group consisting of tuberculosis, candidiasis, aspergillosis, alginosis, nocardia and cryptococcosis.

5. The method of claim 1, wherein the immunity is an immunity against tumor, e.g., solid tumor or leukemia.

6. The method of claim 1, wherein the TGR5-β-arrestin-Src agonist is TGR5 agonist, GRK agonist, β-arrestin agonist, and/or Src agonist,
wherein the GRK agonist is for example an agonist of GRK1, GRK2, GRK3, GRK4, GRK5, and/or GRK6, particularly of GRK2, GKR4, and/or GRK6, more particularly of GRK6, and
wherein the β-arrestin agonist is for example an agonist of β-arrestin-1 and/or β-arrestin-2.

7. The method of claim 1, wherein the TGR5-GRK-β-arrestin-Src agonist is a bile acid source, particularly a bile acid, e.g. primary bile acid or secondary bile acid, unconjugated bile acid or conjugated bile acid.

8. The method of claim 7, wherein the bile acid is selected from the group consisting of cholic acid (CA), chenodeoxycholic acid (CDA), deoxycholic acid (DCA), lithocholic acid (LCA), glycocholic acid, taurocholic acid, glycochenodeoxycholic acid, taurochenodeoxycholic acid, ketolithocholic acid, sulpholithocholic acid, ursodeoxycholic acid, glycodeoxycholic acid, taurodeoxycholic acid, glycolithocholic acid, taurolithocholic acid, and any combination thereof.

9-12. (canceled)

13. The method of claim 1, wherein the enhancing immunity is performed by treating disease treatable by modulating immunity, and wherein the disease is selected from the group consisting of microbial infection including viral infection, bacterial infection and fungal infection, and tumor including solid tumor and leukemia, and the TGR5-GRK-β-arrestin-Src modulatory agent is TGR5-GRK-β-arrestin-Src agonist, e.g., TGR5 agonist, β-arrestin-1/2 agonist, and/or Src agonist.

14. The method of claim 11, wherein the disease is autoimmune disease, and the TGR5-GRK-β-arrestin-Src modulatory agent is TGR5-GRK-β-arrestin-Src antagonist, e.g., TGR5 antagonist, β-arrestin-1/2 antagonist, and/or Src antagonist.

15. The method of claim 1, wherein the enhancing immunity is performed by vaccinating a subject in need thereof, comprising administering to the subject TGR5-GRK-β-arrestin-Src agonist, e.g., TGR5 agonist, β-arrestin-1/2 agonist, and/or Src agonist, as adjuvant separately or in a vaccine composition.

16. The composition of claim 20, which is a vaccination adjuvant composition, comprising TGR5-GRK-β-arrestin-Src agonist, e.g., TGR5 agonist, β -arrestin-1/2 agonist, and/or Src agonist.

17. The composition of claim 20, which is a vaccine composition, comprising TGR5-GRK-β-arrestin-Src agonist, e.g., TGR5 agonist, β-arrestin-1/2 agonist, and/or Src agonist, as adjuvant.

18. A method of identifying immune modulatory agent, comprising
contacting a candidate agent with reporter of TGR5-GRK-β-arrestin-Src pathway, and
determining activity of TGR5-GRK-β-arrestin-Src pathway,
wherein changed activity of TGR5-GRK-β-arrestin-Src pathway indicates the candidate agent is an immune modulatory agent.

19. The method of claim 18, wherein enhanced activity of TGR5-GRK-β-arrestin-Src pathway indicates the candidate agent is an immune enhancer, and reduced activity of TGR5-GRK-β-arrestin-Src pathway indicates the candidate agent is an immune inhibitor.

20. A composition comprising TGR5-GRK-β-arrestin-Src agonist, e.g., TGR5 agonist, β-arrestin-1/2 agonist, and/or Src agonist.
